(19) United States
(12) Patent Application Publication
(10) Pub. No.: US 2008/0118974 A1
(43) Pub. Date: May 22, 2008

(54) Large scale cell culture vessel

(76) Inventors: Gregory Roger Martin, Acton, ME (US); Allison Jean Tanner, Portsmouth, NH (US); Todd M. Upton, Eliot, ME (US)

Correspondence Address: CORNING INCORPORATED, SP-TI-3-1, CORNING, NY, 14831, US

(21) Appl. No.: 11/601,947

(22) Filed: Nov. 20, 2006

(51) Int. Cl. C12M 1/12

(52) U.S. Cl. 435/297.1

(57) ABSTRACT

A multilayered cell culture apparatus for the culturing of cells is disclosed. The cell culture apparatus is defined as an integral structure having a plurality of flexible cell culture compartments in combination with a spacer material maintaining air space(s). The expandable compartments of the cell culture apparatus have imparted therein gas permeable membranes in combination with air spaces that will allow the free flow of gases between the cell culture compartments and the external environment. Furthermore an interconnecting passageway is provided between each compartment and between the cell culturing vessel and the external environment. The expandable vessel promotes the growth of large numbers of adherent or suspension cells by providing the volumes of nutrient medium and gaseous exchange.

### BACKGROUND

1. Field of the Invention

The present invention relates generally to the cellular biological field and in particular, to an expandable cell culture vessel.

2. Technical Background

Culture conditions generally vary depending on the type of cells in culture and the purpose for culturing the cells. The cells may be derived from a number of sources, for example, bacteria, yeast, insects and mammals and may be grown bathed in nutrient medium, in suspension culture or adhered to a substrate. The cells may be used to produce a desired substance, or the cells themselves may be the product.

The ability to use the cells and/or the substances produced from the cell cultures, in part, is determined by the attributes of the vessel supporting the cell culture. The vessel should not contribute to unwanted components in the cell products. As technology advances in the biological field, advances in technological support such as, advances in support equipment must keep pace in order to fully realize the benefit of this knowledge. Cell and gene therapy applications will require cell culture vessels that are able to maintain the strictest aseptic environment, for example, with each cell culture vessel dedicated to growing specific cells for a specific individual.

Currently, a major contributor to the overhead of manufacturing biopharmaceuticals is the cost associated with maintenance, sterilization and validation of non-disposable bioreactors/vessels. Cell culture vessels must be developed to sustain the level of cleanliness required without incurring this excessive expense or risking the possibility of contamination.

Development of optimal process conditions for cell growth usually begins with a single vessel growing thousands of cells, as opposed to monitoring a large-scale system with billions of cells. It is important to maintain the microenvironment of the cells in culture to enable consistent cell performance. Progressing from the single vessel to the large-scale system is not straight-forward, since no simple vessel system exists that permits direct extrapolation of culture conditions from the single vessel to the large-scale system.

Gas exchange between external and internal environments of the cell culturing vessel is necessary in order to sustain the metabolic requirements of the cells in culture. In large-scale systems, special consideration must be given to gas exchange between the external environment and in the nutrient medium within the vessel, since the surface area to volume ratio goes down as the vessel volume increases. This decrease in surface area with increased volume limits the amount of nutrient medium available for diffusion and limits the gas exchange. Spatial gradients can also occur in large-scale vessels, and remedies such as sparging and agitation can lead to foaming of the nutrient medium and also shear damage to the cells in culture.

A number of conventional cell culture systems utilize sheets of polymer to form bags. In U.S. Pat. No. 6,190,913, the polymer sheets are thick enough to withstand wave-like agitation in order to induce mixing and facilitate the gas exchange necessary to ensure proper culture conditions. This equipment requires maintenance and occupies space outside of that needed for the cell culture vessel itself. Other baglike cell culture devices, for example, those disclosed in U.S. Pat. Nos. 4,945,203 and 5,736,398, are formed from polymer sheets that are very thin, thus permitting gas exchange to occur through the polymer sheet material without agitation or sparging. However, handling unsupported, large scale bag vessels is cumbersome and unwieldy.

Alternatively, gas exchange between the external atmosphere and internal culture environment has led to the development of multi-layered high density cell culture vessels as described in, for example, commonly owned U.S. application Ser. No. 11/433,859, the disclosure of which is incorporated herein in its entirety. The vessels described therein are assembled with gas permeable materials and enable these devices to meet the oxygen requirements for cellular metabolism without sparging or agitation. These vessels have an increased surface area for cell growth as well as a suitably rigid structure to permit robotic handling. However, a limitation of these devices occurs because the rigidity that enables robotic handling necessitates that the vessel volume remains fixed, making modifications to culture conditions more problematic.

Thus, there is a need for disposable cell culture vessels which enable the aseptic growth and maintenance of cells in culture utilizing a design that is capable of being scaled from a small volume (<1 L) to a large volume (>10 L) without modifying culture conditions. It would be advantageous to have cell culture vessels which would simplify and reduce the space requirements to perform large scale cell culture operations; in particular, the variables added by perfusion, sparging and movement, as well as the mechanical accessories and equipment needed to perform these manipulations.

Furthermore, there is a need for cell culture vessels, capable of the necessary gas exchange for the growth of cells, which allow for increasing internal volumes of nutrient medium, thus permitting continual growth of multiplying numbers and sizes of cells. This vessel expansion would allow for both the volume increase and augmentation of the nutrient medium during use without withdrawing the cells from their aseptic environment for transfer to a vessel with greater capacity, thus reducing the risk for contamination. It would be advantageous to have such cell culture vessels enable the growth or maintenance of either adhesion dependent or suspension cells in culture. Also, the ability to stack the cell culture vessels would be beneficial in maintaining and expanding large volumes of cells in culture allowing efficient space utilization and ease of handling. Furthermore, there is a need for cost-effective disposable cell culture vessels constructed using optically clear materials for monitoring the growth of cells which may be easily assembled and efficiently utilized.

### SUMMARY OF THE INVENTION

According to one embodiment of the present invention, a cell growth apparatus for efficient culturing of cells is disclosed. The cell growth apparatus comprises a number of compartments, each compartment is expandable and comprises at least one gas permeable, liquid impermeable surface, an interconnection or passageway between each compartment to provide an integral vessel for cellular growth, a spacer to maintain gas exchange positioned between each compartment such that one or more air pockets or spaces are supported there-between.

According to another embodiment of the present invention, an external support structure is provided to support the number of expandable compartments, such that several expandable compartments may be stacked, handled and moved without collapsing the cell growth apparatus. The external support structure reinforces the stability of the composite vessel, cell culture compartments.

In yet another embodiment of the present invention, the external support structure is also expandable to accommodate expanding compartments.

Cell culture vessels and methods using the cell culture vessels of the present invention will actively encourage diffusion and/or dispersion of oxygen within large volumes of cell culture media through gas permeable, liquid impermeable components with increased surface to volume ratios.

Additional features and advantages of the invention will be set forth in the detailed description which follows, and in part will be readily apparent to those skilled in the art from the description or recognized by practicing the invention as described in the written description and claims hereof, as well as the appended drawings.

It is to be understood that both the foregoing general description and the following detailed description are merely exemplary of the invention, and are intended to provide an overview or framework to understanding the nature and character of the invention as it is claimed.

The accompanying drawings are included to provide a further understanding of the invention, and are incorporated in and constitute a part of this specification. The drawings illustrate one or more embodiment(s) of the invention and together with the description serve to explain the principles and operation of the invention.

### BRIEF DESCRIPTION OF THE DRAWINGS

The invention is best understood from the following detailed description when read with the accompanying drawing figures. It is emphasized that the various features are not necessarily drawn to scale. In fact, the dimensions may be arbitrarily increased or decreased for clarity of discussion.

FIG. 1 is a side view of the cell growth apparatus according to one embodiment of the present invention.

FIG. 2 is a top view of one compartment of the cell growth apparatus according to another embodiment of the present invention.

FIG. 3 is a side view of one compartment of the embodiment shown in FIG. 2.

FIG. 4 is a side view of the cell growth apparatus according to another embodiment of the present invention.

FIG. 5 is a side view of the cell growth apparatus according to another embodiment of the present invention.

FIG. 6 is a side view of the cell growth apparatus according to another embodiment of the present invention.

FIG. 7 is a top view of one compartment of the embodiment shown in FIG. 6

FIG. 8 is a side view of the cell growth apparatus according to another embodiment of the present invention.

FIG. 9 illustrates exemplary shapes and porting of the cell growth apparatus of the present invention.

### DETAILED DESCRIPTION

In the following detailed description, for purposes of explanation and not limitation, exemplary embodiments disclosing specific details are set forth in order to provide a thorough understanding of the present invention. However, it will be apparent to one having ordinary skill in the art that the present invention may be practiced in other embodiments that depart from the specific details disclosed herein. In other instances, detailed descriptions of well-known devices and methods may be omitted so as not to obscure the description of the present invention.

Reference will now be made in detail to the present embodiments of the invention, examples of which are illustrated in the accompanying drawings. Wherever possible, the same reference numbers will be used throughout the drawings to refer to the same or like parts.

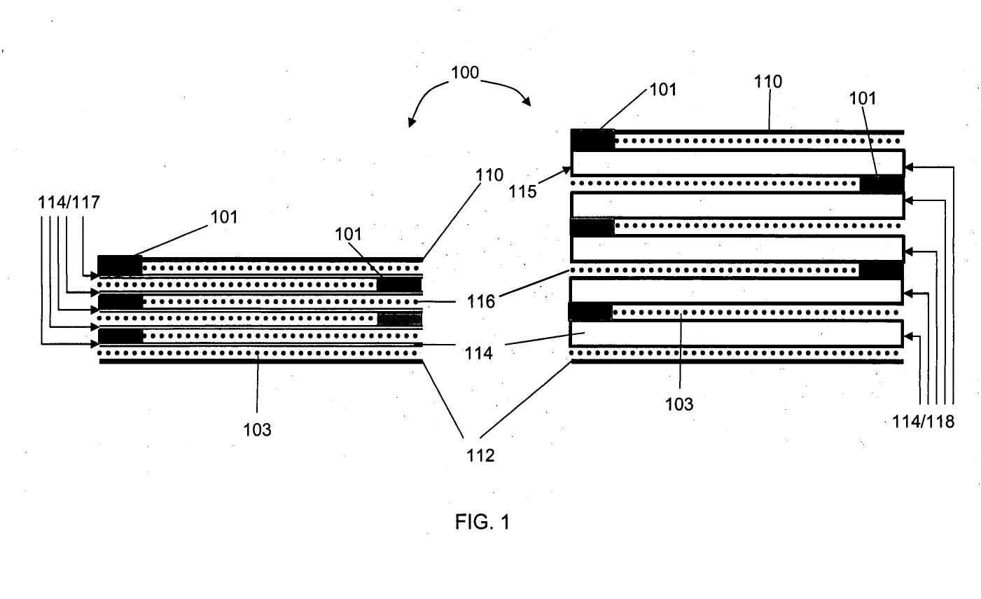

An external view of an apparatus in accordance with one embodiment of the present invention is shown in FIG. 1. The apparatus 100 of this embodiment is a high-density cell culturing device with vertically stacked compartments 114. The compartments 114 are confined by a top platen 110 and a bottom platen 112 composed of a rigid material for various structural configurations having any size, shape, or dimension dependent upon desirable volume capability. Each compartment 114 is made from a gas permeable material sealed together at the edges forming walls 115. Gas permeable compartments 114/117 are collapsed; whereas gas permeable compartments 114/118 are expanded to contain the nutrient medium and growing cells. In this exemplary embodiment, the vessel 100 is comprised of five compartments, though any number of layers/compartments 114 may be stacked as long as the vessel is structurally supported. Ports 101 within each compartment 114 adjoin to the next compartment 114 in sequence and/or provide fluid communication with the external environment. Between each compartment is a spacer material 116 that maintains an area 103 for gas exchange. The compartments are therefore capable of being filled with cells and nutrient media to enclose an expandable volume of cell culture. For example, as the compartments fill with cells and nutrient media, the compartment(s) swell in size from about 1 mm to about 2-5 mm in height to enclose the growing volume of cells. The expansion of the compartment 114 is dependent upon the enclosed volume of liquid, cells and media.

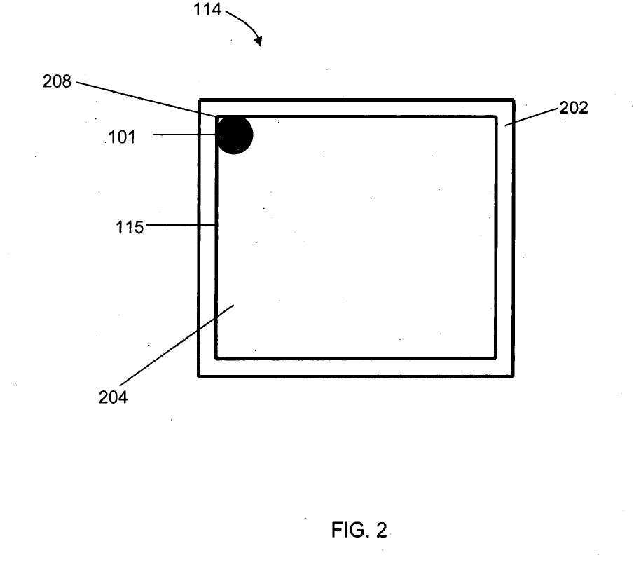

A top view of an exemplary embodiment of one gas permeable compartment 114 of the present invention is illustrated in FIG. 2. The gas permeable compartment 114 in this embodiment is expandable and comprises a compartment frame 202 that provides structural support for a gas permeable compartment 204. The frame 202 itself is supportively rigid and lies outside of the sealed gas permeable material 204 forming a supportive perimeter. A port 101 is designated in one corner 208 of the gas permeable wall 115 which will interconnect with another similar gas permeable compartment 114 in a cell culture stack. Although in this embodiment, the port 101 is shown in the corner of the gas permeable wall 115 through the gas permeable material 204, ports 101 may be located at any location requiring fluid communication into the gas permeable compartments 114 from the external environment, between the gas permeable compartments 114 and/or to the external environment from the gas permeable compartments 114.

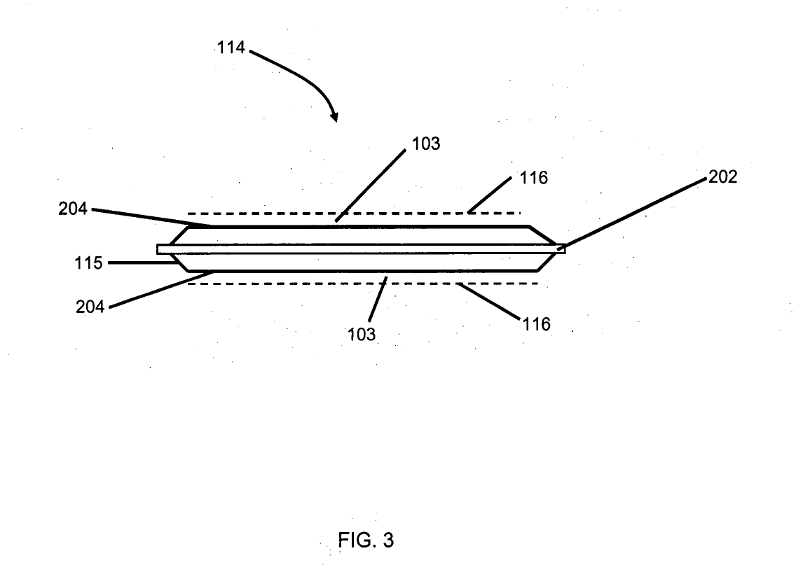

Shown in FIG. 3 is side view of one compartment 114 of the exemplary embodiment shown in FIG. 2. A supportive compartment frame 202 is located outside of the sealed edges 115 of the compartment. A spacer material 116 lies above and below the gas permeable material 204 to maintain air spaces 103 for gases to circulate and diffuse through the gas permeable material 204. The spacer material 116 may be constructed as a planar sheet with features formed therein in order to maintain an air space 103 between gas permeable compartments or between gas permeable compartments and other gas impermeable surfaces. An example of this spacer material is a 0.020 inch thick thermoformed polymer such as polyethylene or polypropylene with rounded pinpoint-size features protruding 0.010 inch from the planar surface in a regular pattern on one or both sides of the planar surface. On the other hand, the spacer material 116 may be any rigid or flexible support network that is capable of maintaining the air space 103 between cell growth compartments 114 to allow for gas diffusion through the air space 103 and between the gas permeable material 204 and the external atmosphere. An example of such a flexible support network is a woven mesh. The construction of the spacer material 116 may be of any composition, for example, polyethylene, polypropylene, polyvinylchloride, polycarbonate, polystyrene, polyester, nylon or other suitable polymer or metal. Suitable metals may be, for example, aluminum, titanium stainless steel or the like. The spacer material may be of any configuration that permits at least a 0.001 inch air space 103 to form between each cell growth compartment 114. Further, any number of compartments 114 may be stacked together with filling and emptying inlets and outlets interconnecting the compartments limited only by the ability of the composite vessel stack to maintain a stable structural conformation. Thus, the present invention can be utilized for adherent or suspension cell culture to promote the growth of large numbers of cells throughout the expandable cell culture vessel 100.

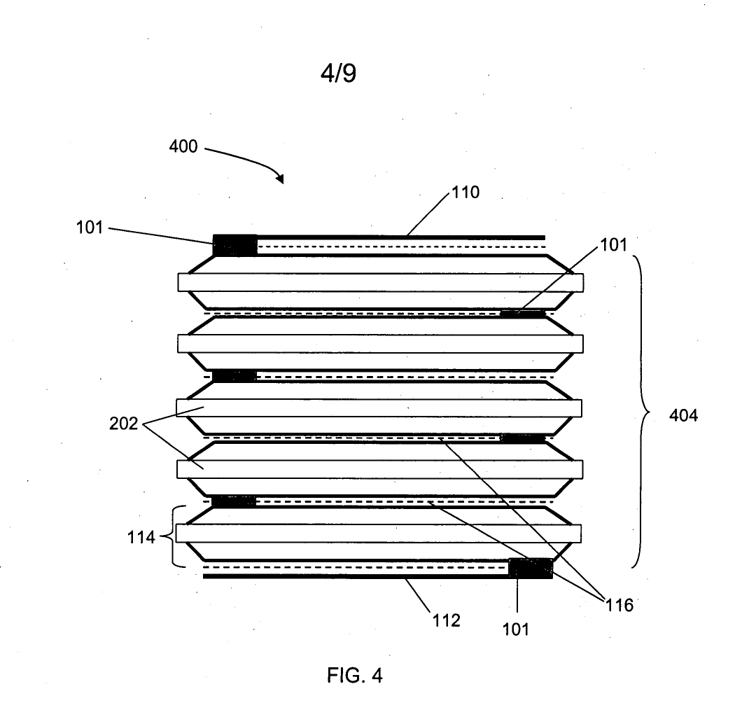

When multiple compartments 114 are stacked, as shown in the exemplary embodiment in FIG. 4, the ports 101 may be staggered for fluid to perfuse through each individual compartment 114 and into the next compartment in series within the five layered vessel 400. Also, the ports 101 may provide entrance and/or exit points for media, fluids, analytical devices and the like. The accordion formation 404 between the top platen 110 and bottom platen 112 including individual flexible compartments 114 formed there-between enables compression and/or expansion of the all of the compartments 114 individually or as a unit depending on the flexibility of each of the individual compartments 114, the rigidity of any of the compartment frames 202, the elasticity of interconnecting ports 101, and/or the pliability of the spacer material 116 which lies between each cell growth compartment 114.

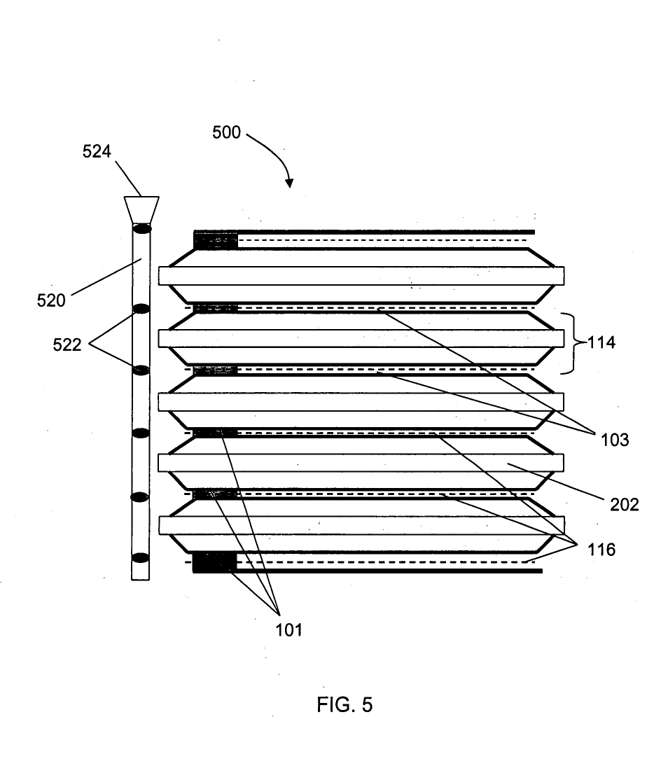

In another embodiment shown in FIG. 5, a large scale cell culture vessel 500 with aligned ports 101. The cell culture vessel 500 is constructed of similar gas permeable materials 204 as in previously discussed embodiments; the gas permeable materials 204 are supported by compartment frames 202, separated by air spaces 103 for gas exchange by the spacer material 116 there-between where fill ports 101 allow fluid communication between each compartment 114. When the ports 101 are aligned, an access tube 520 may be inserted to direct flow of fluid into each port 101. In one aspect, the access tube 520 is a tube with holes 522 corresponding spatially to each port 101 in the cell culture vessel 500. The access tube 520 may be a separate unit as demonstrated in the illustration of FIG. 5 or may be integrally connected and rotated, separately interconnected within the cell culture vessel 500 to permit or restrict flow. Therefore, it would be possible to fill the funnel shaped top 524 of the access tube and seal off other ports 101 to selectively fill specified cell culturing compartments 114.

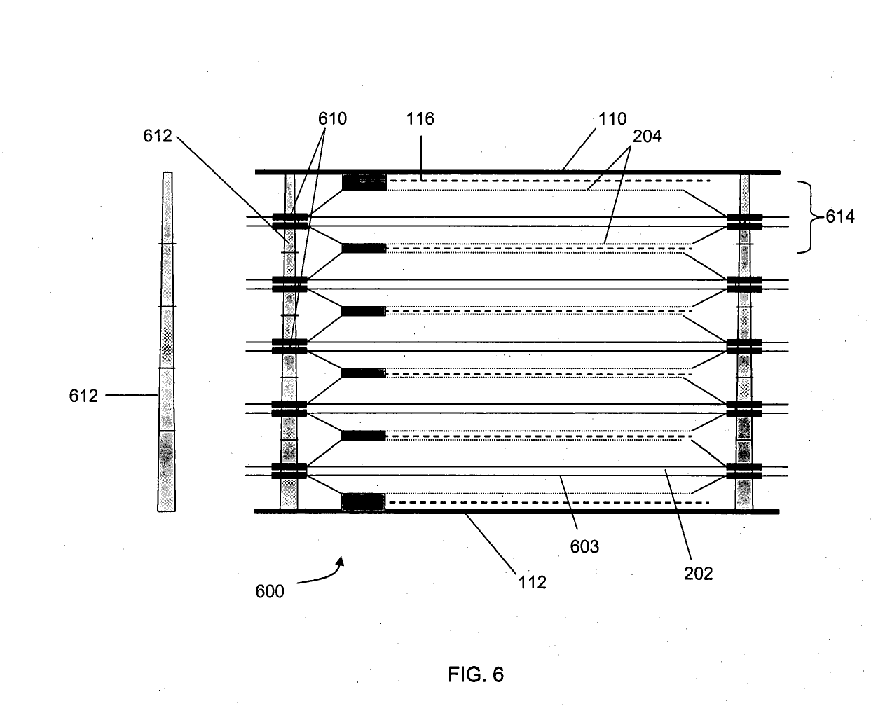

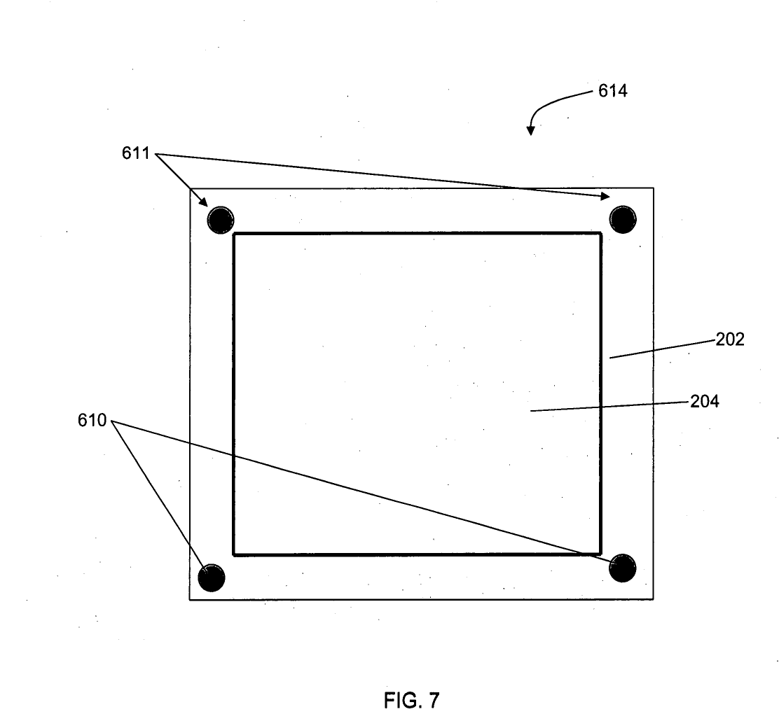

FIG. 6 illustrates one embodiment of a large scale cell culture vessel 600. The vessel 600 includes flexible cell culturing compartments 614 formed between the top platen 110 and the bottom platen 112. The gas permeable materials 204 forming growth surfaces and/or walls of the cell culturing compartments 614 are secured along outer edges 603 of the perimeter of the compartment frame 202. Grommets or other supported hole structures 610 (also illustrated in a top view in FIG. 7) can be positioned through the gas permeable materials 204, the spacer material and the supportive compartment frame at each corner 611 and/or other positions around the perimeter of the cell culturing compartments 614. Support rods 612 which may be telescoping, run through the grommets 610 at the corners 611 and/or other positions around the perimeter of the cell culturing compartment 614 of the stack of compartments and connect onto the rigid top platen 110 and the rigid bottom platen 112. Telescoping support rods are able to collapse or expand with changing volumes within individual compartments or within the entire cell culturing vessel and thusly do not hinder the expanding or collapsing in any way. The support rods 612 assist in providing structural support to the flexible stack of gas permeable cell culturing compartments 614.

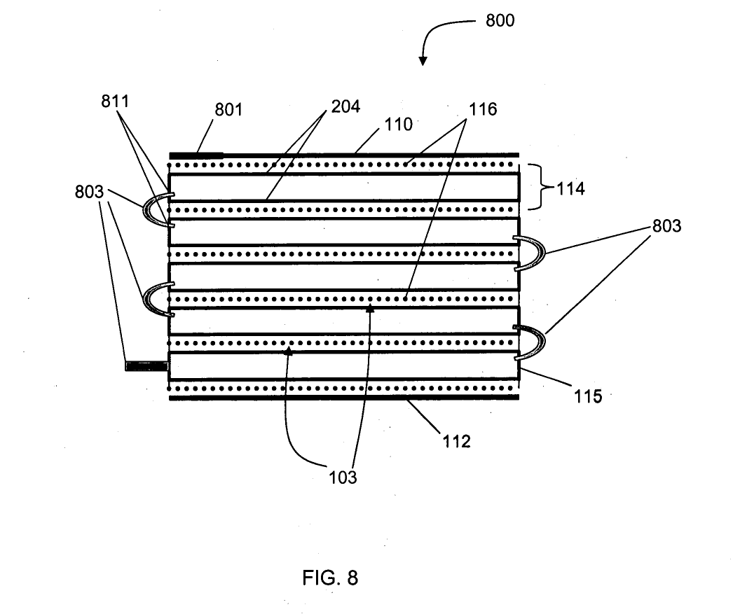

In another embodiment as demonstrated in FIG. 8, each cell culturing compartment 114 between the top platen 110 and the bottom platen 112 is joined by integral tubing 803 formed either by bonding the gas permeable material to form tubes or by attaching preformed tubing. A port 801 of the vessel 800 is included in the rigid top platen 110; however, the port 801 may be incorporated anywhere in a wall of the vessel to allow fluid into and out of the vessel. There may also be multiple ports functioning as either inlets or outlets for accessing one or multiple compartments. The cell culturing vessel 800 is formed by sealing the peripheral edges 115 of two adjacent gas permeable materials 204 to form the individual cell culturing compartments 114 with spacer material 116 to form the air spaces or gaps 103 for gaseous exchange between the individual compartments 114 as well as between the compartments and the rigid external support platens 110/112. The tubing 803 interconnects the ports 811 of each compartment 114; the length and the diameter dimensions of the tubing corresponds to the expansion capacity of the entire vessel, and the viscosity and/or measure of volumetric flow. Moreover, flow rate may be a factor for configuring the design of the vessel.

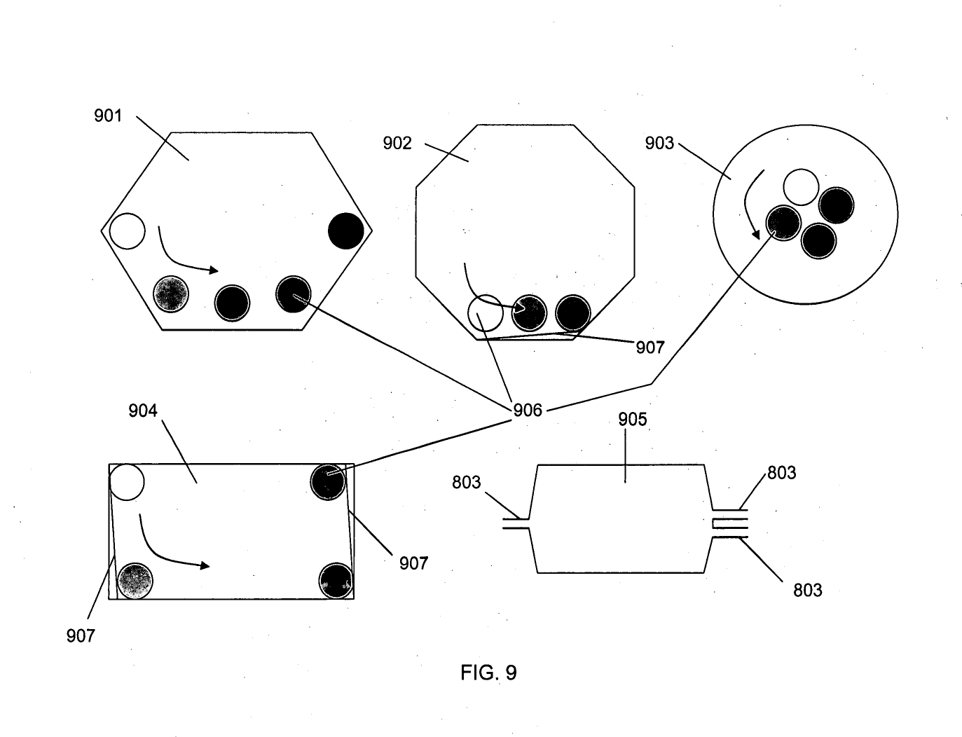

Although any size, shape or configuration of large scale vessel may be utilized, FIG. 9 illustrates some exemplary embodiments of vessel compartment shapes. Each compartment 901/902/903/904 has ports 906 which can have the placement of the ports staggered on successive layers of compartments. The staggering of the ports 906 from one location to another on successively stacked compartments prevents fluid from rushing through the vessel having multiple stacked compartments and permits control of the speed and volume of fluid (media, cell, etc.) flowing into each compartment and prevents the cells from pooling in one location within the compartment or the vessel as they settle. The arrows designate the possible shifted positioning of the ports on successive layers as illustrated by progressively darker shading of inlet/outlet ports 906. A slant 907 in positioning the layers of compartments of the vessel provides the capability of alternating sides by back and forth rocking in order to promote fluid removal. Alternatively, in compartment 905 the ports may be composed of integral tubing 803 located in one or more positions on the perimeter of the compartment 905.

Any material composition useful for culturing cells may be employed in making the compartments of the embodiments of the present invention. The compositions may be durable, flexible or semi-flexible material to allow expansion volumes for cell growth. The composition must be capable of containing a liquid volume and be gas permeable. Possible materials that may be employed to make the cell culture compartments include, but are not limited to, polystyrene, polypropylene, polyethylene, polycarbonate, silicone rubber, fluoroethylenepropylene copolymer, as well as copolymers and multilayers of these materials. Many of these materials are transparent and may be used to make optically clear compartments.

Septa may also be integrally affixed to the body of the apparatus. The septa may take any form well known to those of skill in the art including a slit arrangement useful for blunt needles and as generally described in WO02066595. Possible materials that may be employed in making the septa include natural and synthetic elastomeric materials including, but not limited to fluorocarbon rubber, butyl rubber, polychloroprene rubber, a silicone elastomer composite material, thermoplastic elastomer, medical grades of silicone rubber, polyisoprene, a synthetic isoprene, santoprene and fluoropolymer laminate and combinations thereof. In a preferred embodiment, the elastomeric material is substantially nontoxic to cultured cells. Moreover, the cell culture compartment ports may be joined by a manifold to allow access from a single port and the multiplicity of cell culture compartments may be encased by an external structural support skeleton. Embodiments of the cell culture vessel may therefore be designed to be utilized when significant robotic manipulation is encountered.

The support structure may be made by any number of manufacturing methods well known in the art. Injection molded polymer materials are particularly useful in making the support structure, for example, polystyrene, polypropylene, polyethylene, polycarbonate, silicone rubber, fluoroethylenepropylene copolymer or combinations thereof. One advantage of using polystyrene at a thickness of no greater than 2 mm is that optical clarity through the support structure and through the compartments is maintained. Therefore, cell cultures may be visually monitored from the external environment.

In utilizing the cell culturing apparatus of the present invention, various methods in the industry may be employed in accordance with accepted cell growth culturing. Cells immersed in media are introduced to the vessel through any number of inlets/ports and may be drained via the outlet(s)/port(s). The vessel is arranged such that the cell-containing media covers the cell growth surfaces (e.g. the gas permeable, liquid impermeable surfaces). Advantageously, the cell growth apparatus is capable of being completely filled with media since the gas permeable compartments in combination with the air spaces (as maintained by the spacer material in a flexible/expanding configuration) provide uniform gas distribution to the cell growth surfaces. The spacer, of any material composition, with any porosity or interconnecting arrangement, will further ensure the flow and exchange of gases between the interior of the cell culture compartments and the external environment. If necessary, the vessel may be placed within an area that maintains the appropriate temperature for the particular cells in culture. The cell culturing apparatus may be stacked together with similar cell culturing apparati such that a number of cell cultures are simultaneously grown. The cell culturing apparatus is situated such that the bottom platen or tray assumes a horizontal position. In the case of an adherent cell culture, the cell culturing apparatus can then be inverted to permit the culturing of cells on the opposite surface. Where only gas permeable materials provide the peripheral surfaces for the cell culturing compartments of the cell culturing apparatus, cell growth is enabled on upper and under sides of the compartment (opposing gas permeable surfaces).

During the cell growth process, it may become necessary to extract the exhausted media and insert fresh media. As previously described, media replacement may be achieved through insertion of a canula, for example, through a septum attached to a port, or a port simply uncapped. The convenient construction of the vessel, however, allows the media and/or cells to be drained by opening an outlet port and replaced by directing the media/cells into an inlet port. All of this may be conducted aseptically to avoid risk of contaminating the cultured cells. In the case of attachment dependent cells, once the cells are ready for harvesting, a chemical additive such as trypsin, EDTA and/or other cell release substances may be added to the vessel through the septum. These substances have the effect of releasing the cells from the vessel surfaces. The cells are then harvested from the apparatus. Alternatively, the cells may be released from the surface mechanically, by gentle folding or stretching of the gas permeable surfaces. This enables the cells to be harvested without chemical contribution or damage to the cell structure. Cells in suspension culture may simply be expelled from the vessel along with the nutrient medium for further processing.

As discussed, the embodiments of the present invention are for exemplary purposes only and not limitation. The vessels may be stacked adjacent to one another with platens in direct contact. A diversified network of supports/platens, intersecting and/or alternating gas permeable compartments with spacer material to allow for any number of cell culture compartments and air spaces can be utilized in the embodiments of the present invention so long as they are capable of permitting gas exchange of the cell growth compartments with the external environment. Uniform gaseous distribution throughout the cell culture vessel can therefore be achieved. Furthermore, the apparatus of the present invention may utilize horizontal or vertical designs having surfaces arranged for uniform gaseous distribution to cell growth areas. For convenience, hinged platens may even swing into place to support additional compartments. Clamps may also hold unused compartments closed and rolled-up next to the compartments in use. The flexibility of the vessel components and surfaces therefore provide a variety of options for utilization and design.

The uniformity of conditions for attachment dependent cellular growth may include a determined media volume per unit surface area. Though the determined ratio of volume per unit surface area has previously been known within a confined range of about 0.25-0.5 ml/cm², the ratio is no longer limiting due to the direct access of the cells to gaseous exchange via the gas permeable material surrounding the cells. While efficient use of media is still preferable, any volume of media may be utilized in an apparatus of this invention, the apparatus of which may be any size and/or take any shape suitable for the specified cell growth application. Further, the enhanced capabilities of the present invention may be incorporated in combination with cell growth chambers of standardized or conventionally-sized containers. As stated previously, however, the height and dimensions for cellular growth are no longer restricted so long as an expandable area is included for the growth of cells.

The embodiments of the present invention may be modified to take the shape of any device, container, apparatus, vessel, or flask currently used in industry. Specifically, cylindrical or alternative vessels may utilize gas permeable materials (internal to the vessel) in combination with air spaces to provide an improved culturing environment for the growth of cells. Inclusive in an integral vessel construction are improvements that also incorporate a woven mesh as a spacer material to allow construction of an expansive flexible container. The ability to sparge, perfuse, agitate, or otherwise induce mixing also remain possible with the present invention.

As presented, the multiple embodiments of the present invention offer several improvements over standard vessels currently used in industry. The improved cell culture devices remarkably enhance the number of cells that are capable of being cultured in the volume enclosed by traditional cell culture vessels. The various benefits are attributable to the multi-layered arrangement of gas permeable compartments assembled into a unitary vessel and the semi-rigid construction of those layers. The gas permeable compartment construction alternating with spacer material that creates gas exchange spaces permits continual growth of cells and augmentation of nutrient media volume without necessitating transfer to a larger vessel, thus reducing the risk of contamination. The invention therefore provides an expansion component that is incorporated in the improvement of the cell culturing vessel. The gas permeable compartments further make oxygen and other gases from the external environment available to the internal contents of the apparatus. Specifically, gaseous exchange with the nutrient media is conducive to an even distribution of cell growth when gas permeable materials are utilized in the construction of the cell growth compartments. The cell growth apparatus is capable of fully utilizing its capacity by allowing cells access to optimal volumes of nutrient media and direct oxygenation via the air spaces without the need for cumbersome, space-occupying ancillary equipment. The previously unforeseen benefits have been realized and conveniently offer advantages for exponential cell growth, including a flexible cell culturing apparatus for maintaining gaseous exchange between the internal cell growth areas and external environment as well as an expandable cell culturing apparatus that is designed for easy handling, storage, and accessibility.

As exemplified, the apparatus may include any unitary structure, vessel, device or flask with the capacity to integrally incorporate gas permeable compartments in combination with spacer materials in successive orientation. The invention being thus described, it would be obvious that the same may be varied in many ways by one of ordinary skill in the art having had the benefit of the present disclosure. Such variations are not regarded as a departure from the spirit and scope of the invention, and such modifications as would be obvious to one skilled in the art are intended to be included within the scope of the following claims and their legal equivalents.

It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents.

What is claimed is:

1. A cell growth apparatus comprising:
a plurality of compartments having expandable walls comprising at least one gas permeable, liquid impermeable surface;
a spacer material positioned between each of the compartments to form a gas exchange area there-between;
an interconnecting passageway in fluid communication with each of the compartments and the external environment.

2. The cell growth apparatus according to claim 1, wherein each compartment comprises opposing gas permeable, liquid impermeable surfaces.

3. The cell growth apparatus according to claim 1, wherein each compartment comprises a plurality of gas permeable, liquid impermeable surfaces which form the expandable walls of the compartment.

4. The cell growth apparatus according to claim 1, wherein the gas permeable, liquid impermeable surfaces are selected from the group consisting of polystyrene, polypropylene, polyethylene, polycarbonate, silicone rubber, fluoroethylenepropylene copolymer and a combination thereof.

5. The cell growth apparatus according to claim 1, wherein the spacer material is selected from the group consisting of polyethylene, polypropylene, polyvinylchloride, polycarbonate, polystyrene, polyester, nylon, aluminum, titanium, stainless steel and a combination thereof.

6. The cell growth apparatus according to claim 1, wherein the spacer material is selected from the group consisting of a woven mesh and a planar sheet comprising protruding support features.

7. The cell growth apparatus according to claim 1, further comprising a support structure external to the compartments.

8. The cell growth apparatus according to claim 7, wherein the support structure comprises a compartment frame perimetrically surrounding each of the compartments for supporting the gas permeable, liquid impermeable surfaces.

9. The cell growth apparatus according to claim 7, wherein the support structure comprises a top platen and a bottom platen confining the plurality of compartments there-between.

10. The cell growth apparatus according to claim 7, wherein the support structure is optically transparent.

11. The cell growth apparatus according to claim 9, wherein the support structure further comprises at least one rod inserted through a supported hole in at least one of the compartments and attached to the top platen and the bottom platen.

12. The cell growth apparatus according to claim 10, wherein the rod is telescoping to form an expandable support structure.

13. The cell growth apparatus according to claim 1, wherein the interconnecting passageway comprises ports in the gas permeable, liquid impermeable surface of each compartment.

14. The cell growth apparatus of claim 13, wherein the ports are aligned between two adjacent compartments.

15. The cell growth apparatus of claim 13, wherein the ports are staggered between two adjacent compartments.

16. The cell growth apparatus of claim 13, wherein the interconnecting passageway comprises tubing which connects the port of one compartment to the port of another compartment.

17. The cell growth apparatus of claim 13, further comprising an access tube having holes which correspond spatially to the ports in the gas permeable, liquid impermeable surface of each compartment.

18. The cell growth apparatus according to claim 1, wherein the compartments are optically transparent.

19. A cell growth apparatus comprising:
a plurality of compartments having expandable walls comprising at least one gas permeable, liquid impermeable surface;
a flexible spacer material positioned between each of the compartments to form a gas exchange area there-between;
an interconnecting passageway in fluid communication with each of the compartments and the external environment; and
an expandable support structure external to the compartments to stabilize the cell growth apparatus.

20. The cell growth apparatus according to claim 19, wherein the compartments are optically transparent.
